(19) United States
(12) Patent Application Publication
(10) Pub. No.: US 2025/0381128 A1
(43) Pub. Date: Dec. 18, 2025

(54) CARE AND/OR MAKEUP COMPOSITION COMPRISING ONE OR MORE PIGMENTS

(71) Applicant: L V M H RECHERCHE, SAINT JEAN DE BRAYE (FR)

(72) Inventors: Anny MURA, SAINT JEAN DE BRAYE (FR); Marie HERVÉ-BERDYS, SAINT JEAN DE BRAYE (FR)

(73) Assignee: L V M H RECHERCHE, SAINT JEAN DE BRAYE (FR)

(21) Appl. No.: 18/877,678

(22) PCT Filed: Jun. 21, 2023
(86) PCT No.: PCT/FR2023/050930

§ 371 Date: Dec. 20, 2024

(30) Foreign Application Priority Data
Jun. 21, 2022 (FR) FR2206099

(51) Int. Cl. A61K 8/894; A61Q 1/06

(52) U.S. Cl. CPC A61K 8/894; A61Q 1/06; A61K 2800/31; A61K 2800/5922; A61K 2800/612

(57) ABSTRACT

The present invention relates to a cosmetic composition, preferably an anhydrous cosmetic composition, for caring for and/or making up keratin materials, in particular the skin, eyelids, eyebrows or lips, comprising, in a physiologically acceptable medium: a) a silicone-polyurethane polymer; b) one or more pigments, advantageously treated to be lipophilic, and c) a (C8-C20)alkyl PEG-n Tris(trimethylsiloxy)silyl-(C1-C4)alkyl dimethicone silicone surfactant, where n ranges from 3 to 20, preferably 8 to 12, the (C8-C20)alkyl chain being a saturated, linear or branched, preferably linear, hydrocarbon chain having from 8 to 20 carbon atoms, preferably from 10 to 14 carbon atoms, and the (C1-C4)alkyl chain being a saturated, linear or branched, preferably linear, hydrocarbon chain having from 1 to 4 carbon atoms, preferably 2 carbon atoms.

### FIELD OF THE INVENTION

The present invention relates to the cosmetic field and in particular to cosmetic compositions for caring for and/or making up keratin materials, in particular the skin, eyelids, eyebrows or lips.

### STATE OF THE ART

Skin and/or lip care and/or makeup products intended to beautify and/or color the skin and/or lips are widespread. Products in liquid, semi-fluid or solid form are in particular known. For performance in terms of lastingness (non-transfer) and comfort upon application, film-forming polymers, volatile oils, gelling agents or fatty phase structuring agents, and fillers are generally used to improve the texture and/or sensoriality of the deposit. However, the use of pigments in skin care or makeup compositions, in combination with other ingredients and in particular film-forming polymers, can lead to color inhomogeneities in the compositions, such as color flows or marbling in the compositions.

The Applicant has in particular observed that compositions comprising a silicone-polyurethane polymer and pigments could have color inhomogeneities such as color flows and/or marbling, in particular on stick compositions. It has therefore sought to improve the homogeneity of said compositions and demonstrated that the use of a (C8-C20)alkyl PEG-n Tris(trimethylsiloxy)silyl-(C1-C4)alkyl dimethicone silicone surfactant as defined according to the invention allowed to meet this need.

### PRESENTATION OF THE INVENTION

A first aspect of the invention is therefore a cosmetic composition for caring for and/or making up keratin materials, in particular the skin, eyelids, eyebrows or lips, comprising, in a physiologically acceptable medium:
a) a silicone-polyurethane polymer,
b) one or more pigments, advantageously treated to be lipophilic, and
c) a (C8-C20)alkyl PEG-n Tris(trimethylsiloxy)silyl-(C1-C4)alkyl dimethicone silicone surfactant
   with
   n ranging from 3 to 20, preferably 8 to 12,
   the (C8-C20)alkyl chain being a saturated, linear or branched, preferably linear, hydrocarbon chain having from 8 to 20 carbon atoms, preferably from 10 to 14 carbon atoms, and
   the (C1-C4)alkyl chain being a saturated, linear or branched, preferably linear, hydrocarbon chain having from 1 to 4 carbon atoms, preferably 2 carbon atoms.

Preferably, the composition comprises at least non-white colored pigments (for example iron oxides) and optionally additionally white pigments, as pigments.

Moreover, preferably, the composition of the invention is an anhydrous composition and more preferably it is an anhydrous and solid composition. It also relates to a cosmetic method for caring for and/or making up keratin materials, in particular the skin, eyelids, eyebrows or lips, preferably the lips, comprising the application to said keratin materials, of a composition as defined in the present invention.

### DETAILED DESCRIPTION OF THE INVENTION

The invention therefore relates in particular to a cosmetic composition for caring for and/or making up keratin materials, in particular the skin, eyelids, eyebrows or lips, comprising, in a physiologically acceptable medium:
a) a silicone-polyurethane polymer,
b) one or more pigments, advantageously treated to be lipophilic, and
c) a (C8-C20)alkyl PEG-n Tris(trimethylsiloxy)silyl-(C1-C4)alkyl dimethicone silicone surfactant
   with
   n ranging from 3 to 20, preferably 8 to 12,
   the (C8-C20)alkyl chain being a saturated, linear or branched, preferably linear, hydrocarbon chain having from 8 to 20 carbon atoms, preferably from 10 to 14 carbon atoms, and
   the (C1-C4)alkyl chain being a saturated, linear or branched, preferably linear, hydrocarbon chain having from 1 to 4 carbon atoms, preferably 2 carbon atoms.

‘Keratin materials’ mean in particular the skin of the face, the eyelids, the eyebrows and the lips. According to a particular embodiment, the composition of the invention is applied to the eyelids, the eyebrows or the lips. Preferably, it is applied to the lips.

‘Pigments’, as defined below, mean particles which are white or colored, mineral or organic, insoluble in an aqueous solution, intended to color and/or opacify the resulting deposit. Advantageously in the context of the present invention, the composition will comprise at least (non-white) colored pigments and optionally additionally white pigments.

According to a first embodiment, the composition is a fluid or liquid composition, that is to say a composition which flows under its own weight at room temperature and atmospheric pressure.

According to another particular and preferred embodiment of the invention, the composition of the invention is not a fluid or liquid composition. Thus, the composition of the invention will preferably be a pasty composition or a solid composition.

The rheological properties of a pasty or solid composition according to the invention can be characterized by viscosity/elasticity measurements at 25° C. on the final product.

The viscoelastic parameters of the pasty or solid compositions of the invention are for example measured using a DHR2 rheometer from TA Instruments, at 25° C. and atmospheric pressure (760 mm Hg).

For a ‘solid or pasty composition’ of the invention, a pellet of product is cut and deposited between 2 smooth metal planes of 20 mm diameter and 1 mm air gap. The storage modulus or elastic modulus G′ (expressed in Pascal (Pa)) and the delta phase angle in degrees (°) are recorded as a function of an increasing stress ranging from 0.1 Pa to 10000 Pa. The values retained are extracted from the linear domain in which the moduli are constant, that is to say at deformations of less than 100 Pa.

A product whose storage modulus G′ is greater than 5000 Pa and whose delta phase angle is less than 30° will be considered solid or pasty. Thus, according to a particular and preferred embodiment, a solid or pasty composition of the invention is characterized by a storage modulus G′ greater than 5000 Pa and whose delta phase angle is less than 30°, as measured according to the protocol described above.

According to a particular embodiment, the composition has a storage modulus G′ greater than 20000 Pa, or even greater than 50000 and a delta phase angle will be less than 25°.

According to another particular embodiment, the composition has a storage modulus G′ greater than 100000 Pa, or even greater than 500000 Pa and a delta phase angle will be less than 20°.

According to another embodiment, the solid composition of the invention can be characterized by its hardness. Said hardness can be measured at 20° C. by the method called “butter-cutting wire” method, which consists of transversely cutting a stick of product, which is preferably cylindrical in revolution, using a rigid metal wire with a diameter of 250 μm by moving the wire relative to the stick at a speed of 60 mm/min. The hardness of the samples is expressed in Gram force (Gf) and can be measured using a texturometer of the TAXT Plus Texturometer type. Preferably, the value obtained must be comprised between 140 and 190.

According to a first embodiment, the solid composition of the invention is a stick.

According to another embodiment, the solid or pasty composition of the invention is packaged in a pot or a cup.

According to a particular and preferred embodiment, the composition of the invention is an anhydrous composition.

“Anhydrous” means in particular that water is preferably not added to the compositions but may be present in trace amounts in the various compounds used in the compositions. In particular, the composition according to the invention comprises less than 4% by weight of water, preferably less than 3%, preferably less than 2%, more preferably less than 1%, even more preferably less than 0.5% by weight of water, relative to the total weight of said composition, or even is completely free of water.

Thus, according to a preferred embodiment of the invention, the composition is anhydrous and solid.

### Oily Phase

The oily phase of the composition of the invention comprises at least one oil selected from a hydrocarbon oil, a silicone oil and a mixture thereof, preferably a hydrocarbon oil.

According to a particular embodiment, the hydrocarbon oil is selected from the group consisting of hydrocarbon oils of plant origin, linear or branched C8-C19 alkanes, C₁₀-C₄₀ synthetic ethers, C₁₀-C₄₀ synthetic esters, C₁₂-C₂₆ fatty alcohols, C₁₂-C₂₂ higher fatty acids, and mixtures thereof.

According to a particular and preferred embodiment, the composition of the invention comprises one or more linear or branched C8-C19 alkanes.

As linear or branched C8-C19 alkanes, mention can in particular be made of:
C8-C16 isoalkanes (also called isoparaffins) such as s isododecane, isodecane, isohexadecane, hemisqualane, and for example oils sold under the trade names ISOPAR® or PERMETYL®:
linear alkanes having respectively hydrocarbon chains in
   C9-C17, C10-C14, such as a mixture of undecane and tridecane, marketed by BASF Care Créations under the name Cetiol® Ultimate,
   C15-C19, such as those marketed by Seppic under the name EMOGREEN® L15,
   C9-C12, C12-C14, C16-C18 such as those marketed by BIOSYNTHIS under the name VEGELIGHT® SILK (INCI name C9-12 ALKANE), VEGELIGHT® 1214LC, VEGELIGHT® 1618LC,
   n-dodecane (C12) and n-tetradecane (C14), in particular sold by Sasol under the references PARAFOL® 12-97 and PARAFOL® 14-97 respectively,
and mixtures thereof.

Thus, according to a particular embodiment, the composition comprises one or more linear or branched C8-C19 alkanes selected from the group consisting of branched alkanes such as isododecane, isodecane, isohexadecane, hemisqualane; linear C8-C19 alkanes such as nonane, dodecane, undecane, tridecane, n-tetradecane, n-pentadecane, n-nonadecane and mixtures thereof, in particular isododecane and mixtures of linear C9/C12, C11/C13, C10/C12, C10/C14, or C15/C19 alkanes.

According to a particular embodiment, the composition comprises at least isododecane as a C8-C19 branched alkane.

According to a particular embodiment, the total content of linear or branched C8-C19 alkanes in the composition of the invention will be at least 30% by weight relative to the total weight of the composition. In particular, it will range from 40 to 80% by weight, in particular from 45 to 60% by weight relative to the total weight of the composition.

### Silicone-Polyurethane Polymer

The composition of the invention comprises a silicone-polyurethane polymer.

‘Silicone-polyurethane polymer’ according to the invention means any polymer comprising organosiloxane units and urethane bonds.

According to a particular embodiment, the silicone-polyurethane polymer according to the invention is the reaction product of a polyorganosiloxane functionalized by hydroxyl groups, preferably comprising at least two hydroxyl groups, with a diisocyanate compound.

Such a polyorganosiloxane functionalized by hydroxyl groups corresponds to the formula (I)

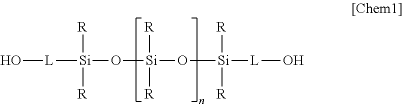

wherein R is independently selected at each occurrence from a hydrogen atom, a hydroxyl group, and optionally substituted hydrocarbon groups containing from 1 to 10 carbon atoms, and in particular from a substituted or unsubstituted alkyl, alkenyl, alkynyl, aryl, aryl-alkyl or alkyl-aryl group; preferably, R is selected from linear, cyclic or branched and optionally substituted C1-C6 alkyl or alkenyl groups, including, without limitation, methyl, ethyl, propyl, isopropyl, butyl, isobutyl, t-butyl, amyl, hexyl, cyclohexyl, vinyl, allyl or C1-C8 aryl, aryl-alkyl or alkyl-aryl groups, including, without limitation, phenyl, benzyl, tolyl, xylyl,
wherein each of the above R groups may be optionally substituted with one or more heteroatoms, including oxygen, nitrogen, phosphorus and halogen, particularly fluorine, as exemplified by fluoroalkyl (perfluoroalkyl) groups such as mono-, di- and trifluoromethyl, perfluorophenyl, and C1-C6 substituted alkyl amino groups, including those having the formula —(CH₂)₁₋₆—NR^{N}₂ and —(CH₂)₁₋₆—NR^{N}—(CH₂)₁₋₆—NR^{N}₂ where R^{N} is typically hydrogen, but may also be methyl, ethyl, propyl, and the like; polyether groups, including, without limitation, polyethylene oxide groups corresponding to the formula —(CH₂CH₂O)ₙ—, propylene oxide groups corresponding to the formula —(CH(CH₃)CH₂O)ₙ— and combinations thereof; and amine, phosphate, hydroxyl, ester and/or carboxylate oxide functions, or the like; or
wherein R may comprise an additional group —L—OH;
wherein L represents either a bond or a linking group; preferably L is a linking group selected from divalent hydrocarbons having from 1 to 10 carbon atoms, including a divalent alkyl, alkenyl, alkynyl, aryl, alkyl-aryl or aryl-alkyl group, such as for example a C1-C10 alkyl group, including, without limitation, divalent groups of the formula —(CH₂)₁₋₁₀—, preferably (CH₂)₁₋₆—, and more preferably, L is —CH₂CH₂CH₂—;
and where n is an integer between 0 and 5000, preferably between 1 and 200, more preferably between 10 and 100, and even more preferably between 10 and 50.

Preferably, R represents at least one or more occurrences of a methyl group, more preferably, R represents a methyl group at all or almost all of the occurrences, which means that R represents a methyl group at more than 90% of the occurrences, in particular at more than 95% of the occurrences, or even at more than 98% of the occurrences. According to one embodiment of the invention, the polyorganosiloxane functionalized by hydroxyl groups comprises a polymethylsiloxane corresponding for example to the structure of formula Ia:

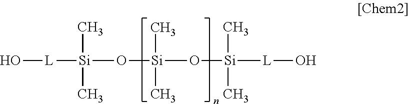

wherein L and n are as defined above.

According to a preferred embodiment of the invention, the polyorganosiloxane functionalized by hydroxyl groups comprises a polymethylsiloxane corresponding for example to the structure of formula Ib:

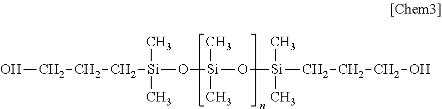

where n is as defined above.

Diisocyanate may in particular correspond to the formula O═C═N—R¹—N═C═O, where R¹ is a divalent hydrocarbon group containing from 1 to 20 carbon atoms, optionally including 20 substituted by one or more heteroatoms, and in particular R¹ may be selected from a cyclic alkyl, linear, cyclic or branched, alkenyl, alkynyl, aryl, alkyl-aryl or arylalkyl group, optionally substituted, including, without limitation:
i. a group of formula:

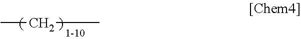

ii. a group of formula:

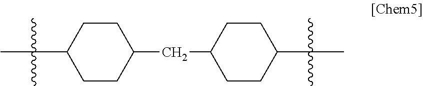 [SMILES: *C1CCC(CC2CCC(*)CC2)CC1]

iii. a group of formula:

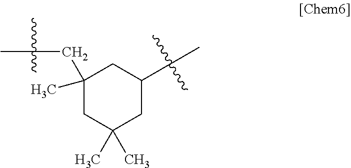 [SMILES: *CC1(C)CC(*)CC(C)(C)C1]

iv. a group of formula:

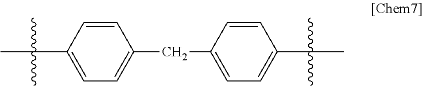 [SMILES: *c1ccc(Cc2ccc(*)cc2)cc1]

v. a group of formula:

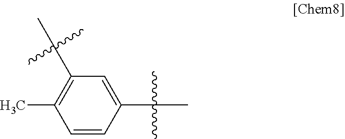 [SMILES: *c1ccc(C)c(*)c1]

and combinations thereof.

Diisocyanates suitable for use in the context of the invention comprise, without limitation, toluene diisocyanate; methylene diphenyl diisocyanate, including 2,2′-MDI, 2,4′-MDI and 4,4′-MDI; 1,6-hexamethylene diisocyanate; isophorone diisocyanate; dicyclohexylmethane 4,4′-diisocyanate; xylene diisocyanate; cyclohexane diisocyanate; 3,3′-dimethyl-4,4′-diphenylmethane diisocyanate; p-phenylene diisocyanate; m-phenylene diisocyanate; 4,4′-isopropylidene dicyclohexyl isocyanate; and equivalents thereof.

In a preferred embodiment, diisocyanate is selected from the group consisting of 1,6-hexamethylene diisocyanate, dicyclohexylmethane 4,4′-diisocyanate, isophorone diisocyanate, and combinations thereof.

In one embodiment, diisocyanate comprises or consists essentially of 1,6-hexamethylene diisocyanate.

In another embodiment, diisocyanate comprises or consists essentially of isophorone diisocyanate.

In yet another embodiment, diisocyanate comprises or consists essentially of dicyclohexylmethane 4,4′-diisocyanate, this embodiment being particularly preferred.

Thus, according to a particular embodiment, the silicone-polyurethane polymer is the reaction product
of a polyorganopolysiloxane functionalized by hydroxyl groups, in particular corresponding to the structure of formula (I)

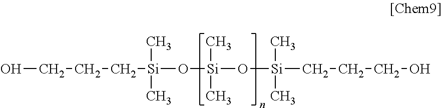

wherein n is an integer between 0 and 5000, preferably between 1 and 200, more preferably between 10 and 100, and even more preferably between 10 and 50,
with a diisocyanate compound, in particular selected from the group consisting of 1,6-hexamethylene diisocyanate, dicyclohexylmethane 4,4′-diisocyanate, isophorone diisocyanate, and combinations thereof.

According to a preferred embodiment, the silicone-polyurethane polymer according to the invention comprises repeating units derived from polyorganosiloxane functionalized by hydroxyl groups and from diisocyanate in the form of an alternating copolymer AB, where unit A has the structure of formula II:

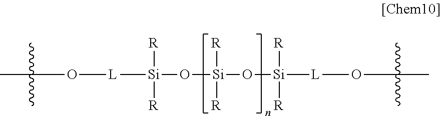

wherein R, L and n are defined as above in relation to formula I, Ia, Ib and Ie, and where unit B has the structure of formula III:

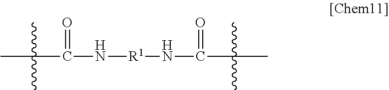

where R1 is as defined above, and wherein units A and B are arranged in a linear, branched or cyclic, and preferably linear, configuration.

The polymer may also comprise branching or grafting points in the polyorganosiloxane wherein one or more groups R in formula I or II is a group IV such that:

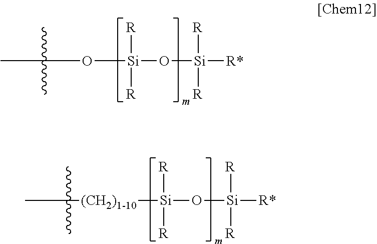

wherein R is as defined for formula I, and R* may represent a —L—O— group further coupling the side chain of a unit B of formula III, which may in turn be further coupled to unit A of formula II, and so on, or R* may represent —L—OH, a group R as defined above, or a terminal group.

When the polyorganosiloxane has such branching or grafting points, they may be present as T or Q type grafts, where T indicates that only one group R on the atom Si is a polyorganosiloxane chain, as shown above, and Q indicates that both groups R are polyorganosiloxanes.

These types of polyorganosiloxane compounds are called copolymer of silicone-polyurethane polymer and resin T or resin Q, which is branched or grafted.

Silicone-polyurethane polymers can also be prepared from functionalized isocyanate prepolymers. For example, an isocyanate prepolymer can be a difunctional or multifunctional polyorganosiloxane isocyanate, such as the polyorganosiloxane diisocyanate shown below in Formula V:

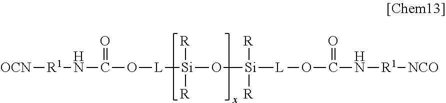

wherein R, R¹ and L are as defined above, and where x is an integer between 0 and 5000, preferably between 1 and 200, more preferably between 10 and 100, and even more preferably 10 and 50.

The prepolymer can also be multifunctional by introducing additional isocyanate groups carrying one or more groups R. The functionalized isocyanate prepolymer is reacted with a polyorganosiloxane functionalized by hydroxyl groups to obtain a compound of formula I or a multifunctional analog thereof.

The prepolymer according to formula V generally has a molecular weight ranging from 4000 to about 15000 Daltons. The prepolymer according to formulas I, Ia, Ib and typically has a molecular weight ranging from 250 to about 15000 Daltons.

In one embodiment of the invention, the silicone-polyurethane polymer is free or essentially free of polyalkylene glycol, especially polyethylene glycol (PEG) or polypropylene glycol (PPG) subunits.

“Essentially free of” means that the polymer comprises less than about 1% by weight, preferably less than about 0.5% by weight, and more preferably less than about 0.1% in 20% by weight of polyalkylene glycol subunits.

In a preferred embodiment, the silicone-polyurethane polymer used in the cosmetic compositions of the invention is a linear polymer comprising the reaction product of formula Ib with a diisocyanate selected from the group consisting of 1,6-hexamethylene diisocyanate, dicyclohexylmethane 4,4′-diisocyanate, isophorone diisocyanate, and combinations thereof.

Such a silicone-polyurethane polymer is for example available from Siltech Corporation in the form of a premix in isododecane, under the commercial reference SILMER UR 5050 or UR 100100 (45% polymer dry matter in isododecane).

According to another embodiment, a silicone-polyurethane polymer is used in the form of a premix in a hemi-squalane.

According to another embodiment, a silicone-polyurethane polymer is used in the form of a premix in a linear C9-C16 alkane or a mixture of linear C9-C16 alkanes, preferably a mixture of n-nonane and n-dodecane (C9-C12, VEGELIGHT SILK) or a mixture of n-undecane and n-tridecane (C11-C13, CETIOL ULTIMATE).

According to a particular and preferred embodiment, the silicone-polyurethane polymer according to the invention is present in the composition of the invention in a content ranging from 3 to 25%, in particular from 5 to 20%, in particular from 8% to 15% by weight of active material (dry matter) relative to the total weight of the composition.

### Coloring Materials

The composition of the invention further comprises coloring materials, and preferably at least pigments as described below.

For the purposes of the present invention, the term “coloring material” means a compound capable of producing a colored optical effect when formulated in sufficient amount in a suitable cosmetic medium. In particular, the colored optical effect is related to the presence of (non-white) colored particles in the composition.

A coloring material may be selected from organic or inorganic coloring materials, optical effect materials, and mixtures thereof.

According to a particular embodiment, the coloring material(s) are pigments, in particular selected from mineral pigments, organic pigments, composite pigments, pearlescent pigments and mixtures thereof.

“Pigments” mean white or particles which are colored, mineral or organic, insoluble in an aqueous solution, intended to color and/or opacify the resulting deposit.

Preferably, and in view of the problem of color flow or marbling solved by the present invention, the composition of the invention comprises at least (non-white) colored pigments, and also in addition white pigments.

Mention can be made of mineral pigments, organic pigments, composite pigments (that is to say pigments based on mineral and/or organic materials), pearlescent pigments or pearlescents (multi-layer structures), and mixtures thereof.

Among the “mineral pigments”, mention may be made, as examples, of titanium dioxide (rutile or anatase), optionally surface-treated; black, yellow, red and brown iron oxides; manganese violet; ultramarine blue, chromium oxide, hydrated chromium oxide and ferric blue. According to a particular and preferred embodiment, the composition of the invention will comprise at least colored pigments, in particular iron oxides, and advantageously in addition white pigments, such as titanium dioxide. Preferably, use will be made of titanium dioxide; black, yellow, red and brown iron oxides and mixtures thereof.

Examples of “organic pigments” include pigments D & C red nº 19; D & C red nº 9; D & C Red nº 22; D & C Red nº 21; D & C Red nº 28; D & C Yellow nº 6; D & C orange nº 4; D & C orange nº 5; D & C Red nº 27; D & C red nº 13; D & C Red nº 7; D & C Red nº 6; D & C Yellow nº 5; D & C Red nº 36; D & C Red nº 33; D & C orange nº 10; D & C yellow nº 6; D & C Red nº 30; D & C red nº 3; D & C Blue nº 1; carbon black and lacquers.

Among the “pearlescent pigments” or “pearlescents”, mention may be made in particular of white pearlescent pigments, such as bismuth oxychloride; and colored pearlescent pigments, such as titanium mica with iron oxides, titanium mica with an organic pigment of the aforementioned type, as well as pigments based on bismuth oxychloride. Mention may be made of the ranges of commercial reference pearlescents Reflecks®, Ronastar®, Timiron® and Syncristal®. The pearlescent pigments may also have a base of silica, alumina, alumina hydroxide, synthetic mica, tin oxide, borosilicate (for example: calcium sodium borosilicate or calcium aluminum borosilicate).

Pigments can be treated or not.

According to a particular embodiment, the pigments are treated with a lipophilic or hydrophobic surface agent.

In particular, lipophilic agents selected from the group consisting of N-acylated amino acids or their salts, silicone agents, metallic soaps, lecithin and its derivatives, waxes, fatty esters, and phospholipids may be mentioned.

Thus, according to a particular embodiment, the composition of the invention comprises mineral pigments treated with a lipophilic surface agent, in particular titanium dioxide and iron oxides treated with N-acylated amino acids or their salts.

The composition of the invention may further comprise dyes, in addition to the pigments of the aforementioned types.

By “dyes” mention can in particular be made of Yellow 5, Yellow 6, Blue 1, Green 5, Green 3, Green 6, Orange 4, Red 4, Red 21, Red 22, Red 27, Red 28, Red 33, Red 40. Liposoluble dyes are for example Sudan red, D&C Red 17, D&C Green 6, beta-carotene, Sudan brown, D&C Yellow 11, D&C Violet 2, D&C Orange 5, quinoline yellow, annatto.

According to a particular and preferred embodiment, the total content of coloring material in the composition, in particular the total content of pigments, will range from 2 to 20% by weight, preferably from 3% to 15% by weight relative to the total weight of the composition.

Preferably, the content of non-white colored pigments will range in particular from 1 to 15%, in particular from 2 to 10% by weight relative to the total weight of the composition.

### Silicone Surfactant

‘Silicone surfactant’ or ‘silicone emulsifier’ will be considered indifferently in the description.

The composition of the invention further comprises a (C8-C20)alkyl PEG-n Tris(trimethylsiloxy)silyl-(C1-C4)alkyl dimethicone silicone surfactant
with
n ranging from 3 to 20, preferably 8 to 12,
the (C8-C20)alkyl chain being a saturated, linear or branched, preferably linear, hydrocarbon chain having from 8 to 20 carbon atoms, preferably from 10 to 14 carbon atoms, and
the (C1-C4)alkyl chain being a saturated, linear or branched, preferably linear, hydrocarbon chain having from 1 to 4 carbon atoms, preferably 2 carbon atoms.

For the purposes of the invention, ‘n’ represents the number of ethyloxy units in the PEG chain.

According to a particular embodiment, the silicone surfactant used according to the invention is Lauryl PEG-10 Tris(trimethylsiloxy)silylethyl Dimethicone, such as that marketed under the name DOWSIL™ ES-5300 by the company Dow Chemical.

The Applicant has in fact shown that the introduction of a (C8-C20)alkyl PEG-n Tris(trimethylsiloxy)silyl-(C1-C4)alkyl dimethicone silicone surfactant as defined above allows to improve the compatibility of the silicone polyurethane polymer and the pigments in a composition according to the invention. Thus the formula is homogeneous, it no longer has any flow and/or marbling, which is not the case without this particular silicone surfactant.

The content of (C8-C20)alkyl PEG-n Tris(trimethylsiloxy)silyl-(C1-C4)alkyl dimethicone silicone surfactant as described above in the composition of the invention will range in particular from 0.1 to 3%, in particular from 0.5 to 2% by weight relative to the total weight of the composition.

### Additional Ingredients

The composition will further advantageously comprise fatty phase structuring agents, in particular selected from waxes, pasty fatty substances and mixtures thereof.

Thus, according to a particular and preferred embodiment, the composition further comprises one or more waxes, in particular one or more waxes of plant origin, and optionally additionally one or more pasty fatty substances.

### Waxes

According to a particular and preferred embodiment, the composition of the invention will further comprise at least one wax, allowing to stabilize, or even freeze the system without disturbing the lastingness result.

Within the meaning of the present invention, the term “wax” means a compound that is solid at 25° C. and that has a reversible solid/liquid state change and a melting point greater than or equal to 40° C., preferably greater than 60° C.

Mention can be made of natural waxes (sunflower wax, carnauba wax, beeswax), microcrystalline waxes, paraffin waxes, polyethylene waxes, ozokerite, and mixtures thereof.

Preferably, the fatty phase of the composition of the invention is structured by one or more waxes of plant origin, in particular selected from the group consisting of carnauba wax, sunflower wax, and a mixture thereof. It may also comprise other additional waxes, such as beeswax, rice wax, candelilla wax, soybean wax, rapeseed wax, olive wax, cotton wax, Chinese insect wax, montan wax, kapok wax, sugar cane wax, jojoba wax, shellac wax, a behenic ester of hydrogenated castor oil fatty acid, C18-C36 fatty acid triglycerides or a mixture thereof.

Thus, according to a particular embodiment, the composition will comprise a carnauba wax, a sunflower wax and a beeswax.

A composition according to the invention may comprise a total wax content ranging from 0.5% to 25% by weight, in particular from 3 to 25% by weight relative to the total weight of the composition.

According to a particular and preferred embodiment, the composition of the invention comprises a total content of wax(es) ranging from 5 to 25% by weight, in particular from 10 to 20% by weight relative to the total weight of the composition.

### Pasty Fatty Substance

The composition may further advantageously comprise one or more pasty fatty substances.

“Pasty fatty substance” means a non-crystalline fatty compound comprising, at a temperature of 25° C., a liquid fraction and a solid fraction.

Examples of pasty fatty substances include, in particular, fatty acid triglycerides and their derivatives, esters of polyol(s) and of dimer diacid of fatty acid, or of one of its esters, hydrogenated castor oil esters, glycerol oligomeric esters, in particular diglycerol esters, vegetable butters and mixtures thereof.

Advantageously, pasty fatty substances of natural origin, preferably of plant origin will be used.

According to a particular embodiment of the invention, the composition will comprise at least one pasty fatty substance selected from jojoba esters. The total content of pasty fatty substances may range from 0 to 15%, in particular from 1 to 10%, in particular from 2 to 8% by weight relative to the total weight of the composition.

According to a particular and preferred embodiment of the invention, the composition of the invention will comprise at least one wax of plant origin selected from a sunflower wax, a carnauba wax, and a mixture thereof, and advantageously in addition a pasty fatty substance, in particular selected from jojoba esters.

A particular composition of the invention will comprise at least one wax of plant origin selected from a sunflower wax, a carnauba wax, and a mixture thereof, and advantageously in addition a beeswax and a pasty fatty substance, in particular selected from jojoba esters.

According to a particular embodiment of the invention, the composition further comprises another additional ingredient selected from lipophilic gelling agents, fillers, and mixtures thereof.

### Lipophilic Gelling Agent

“Lipophilic gelling agent” according to the invention means a compound capable of gelling the fatty phase of a composition.

Mention can in particular be made of mineral gelling agents such as bentones dispersed in an oil. For example, mention can be made of the reference BENTONE GEL ISD V from Elementis with the INCI name Isododecane (and) Disteardimonium Hectorite (and) Propylene Carbonate.

Thus, according to a particular embodiment, the composition of the invention further comprises a lipophilic gelling agent, in particular a mineral gelling agent such as bentones, preferably a quaternary hectorite.

The total content of lipophilic gelling agent(s) may range from 0 to 5%, in particular from 0.5 to 5%, in particular from 2 to 4% by weight relative to the total weight of the composition.

### Fillers

Fillers are used in particular to modify the rheology or texture of the composition and/or to provide a mattifying effect.

For the purposes of the invention, the term “fillers” means particles of any shape, colorless or white, of mineral or organic, natural or synthetic nature, which are in a form (platelet, spherical or oblong), insoluble and dispersed in the medium of the composition. The fillers are chosen in particular from micas, of natural or synthetic origin, kaolin, titanium oxides; calcium carbonate, magnesium carbonate and hydrocarbonate; zinc, magnesium or lithium stearate, zinc laurate, magnesium myristate; synthetic polymer powders, such as polyethylene, polyesters, polyamides (for example nylon); polyacrylic or polymethacrylic acid powders, silicone powders; cellulose powders; glass and ceramic beads; powders of organic materials of natural origin such as corn, wheat, rice, cotton starches, cross-linked or not, and mixtures thereof.

The fillers may or may not be surface treated, in particular with a lipophilic or hydrophobic agent.

According to a particular embodiment, the filler is not surface treated.

According to a particular and preferred embodiment, the filler is surface-treated, in particular with a lipophilic or hydrophobic agent. Mention may in particular be made of lipophilic agents selected from the group consisting of N-acylated amino acids or their salts, silicone agents, metallic soaps, lecithin and its derivatives, waxes, fatty esters, and phospholipids. According to a particular and preferred embodiment, the composition of the invention comprises at least one silica powder, preferably a silica powder surface-treated with a lipophilic or hydrophobic agent.

Preferably, the silica powder is treated with a lauroyl lysine. In particular, mention can be made of the commercial reference AMILON® from the company IKEDA.

According to a particular embodiment, the composition advantageously comprises a silica powder treated with a lauroyl lysine and an untreated silica powder.

According to a particular embodiment, the silica powder, in particular the hydrophobically treated silica powder, is present in the composition in a content ranging from 2 to 10%, in particular from 3 to 8% by weight relative to the total weight of the composition.

According to a particular embodiment, the total content of filler(s) in the composition will range in particular from 2 to 10%, in particular from 3 to 8% by weight relative to the total weight of the composition.

Thus, according to a particular and preferred embodiment of the invention, the composition of the invention comprises:
one or more hydrocarbon oils, in particular isododecane and linear C9-C14 alkanes,
a silicone-polyurethane polymer produced from the reaction (i) of a polyorganopolysiloxane functionalized by hydroxyl groups corresponding to the structure of formula (I)

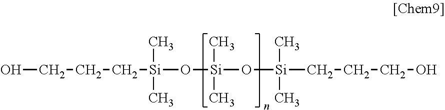

wherein n is an integer between 1 and 200, more preferably between 10 and 100, and more preferably still between 10 and 50, with (ii) a diisocyanate compound, in particular selected from the group consisting of 1,6-hexamethylene diisocyanate, dicyclohexylmethane 4,4′-diisocyanate, isophorone diisocyanate, and combinations thereof.
lipophilic treated pigments, in particular lipophilic treated iron oxides,
a (C8-C20)alkyl PEG-n Tris(trimethylsiloxy)silyl-(C1-C4)alkyl dimethicone silicone surfactant as defined above, preferably Lauryl PEG-10 Tris(trimethylsiloxy)silylethyl Dimethicone,
at least one wax, preferably of plant origin, in particular selected from sunflower wax, carnauba wax and a mixture thereof,
at least one pasty fatty substance, in particular a jojoba ester,
optionally in addition at least one lipophilic gelling agent, in particular a bentone, and
at least one filler, in particular a silica, advantageously treated to be lipophilic.

According to a preferred embodiment, said composition comprises at least non-white colored pigments (for example: iron oxide), and optionally additionally white pigments (for example: titanium dioxide).

According to a particular and preferred embodiment, said composition is an anhydrous and solid composition as described above.

### Galenic

The composition of the invention is a care and/or makeup composition for keratin materials, in particular a composition for the skin, eyelids, eyebrows, or lips, preferably the eyelids, eyebrows and lips.

It can be a fluid or liquid composition, or a solid or pasty composition.

Depending on the particular embodiment, the composition is fluid or liquid. It can be in particular a liquid or fluid foundation.

Preferably, it is a solid or pasty composition. It may be in particular a stick, a pencil or a composition packaged in a pot or a cup.

According to a first embodiment, the composition of the invention is a “top-coat” composition, which can be used on any lip makeup composition, to provide a long-lasting matte result (transfer-free).

According to another embodiment, the composition of the invention is a one-step makeup composition, to provide color, matte finish and lastingness (non-transfer).

In particular, it will be a lip stick or a composition in a pot or cup for the lips.

In another particular embodiment, it will be a liner pencil.

According to another particular embodiment, it will be a stick or a pencil or a composition in a pot or cup, applicable with the finger or a brush, for the eyebrows (for example: eyebrow pencil).

In another embodiment, it will be a stick or a composition in a pot or cup, applicable with the finger, sponge or brush, for the eyelids (for example: eye shadow).

In another embodiment, it will be a stick or a composition in a pot or cup, applicable with the finger, sponge or brush, for the skin of the face, in particular certain areas (for example: complexion corrector or concealer).

Thus, the composition of the invention is in particular in the form of a stick or a composition packaged in a pot or a cup, a liner pencil, an eyebrow pencil, an eye shadow, or a concealer, preferably a lip stick.

### Cosmetic Method

The invention also relates to a cosmetic method for caring for and/or making up keratin materials comprising the application, to said keratin materials, in particular the skin, the eyelids, the eyebrows, or the lips, preferably the lips, of a composition according to the invention.

The compositions of the invention applied to the skin or lips are as described above.

Preferably the composition is an anhydrous composition.

More preferably, it is a pasty or solid anhydrous composition as defined above.

According to a particular embodiment, the composition of the invention applied to the skin or the lips, preferably the lips, comprises:
one or more hydrocarbon oils, in particular isododecane and linear C9-C14 alkanes,
a silicone-polyurethane polymer produced from the reaction (i) of a polyorganopolysiloxane functionalized by hydroxyl groups corresponding to the structure of formula (I)

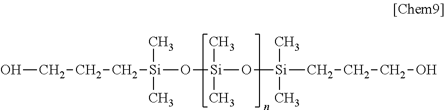

wherein n is an integer between 1 and 200, more preferably between 10 and 100, and more preferably still between 10 and 50, with (ii) a diisocyanate compound, in particular selected from the group consisting of 1,6-hexamethylene diisocyanate, dicyclohexylmethane 4,4′-diisocyanate, isophorone diisocyanate, and combinations thereof.
lipophilic treated pigments, in particular lipophilic treated iron oxides,
a (C8-C20)alkyl PEG-n Tris(trimethylsiloxy)silyl-(C1-C4)alkyl dimethicone silicone surfactant as defined above, preferably Lauryl PEG-10 Tris(trimethylsiloxy)silylethyl Dimethicone,
at least one wax, preferably of plant origin, in particular selected from sunflower wax, carnauba wax and a mixture thereof,
at least one pasty fatty substance, in particular a jojoba ester,
optionally in addition at least one lipophilic gelling agent, in particular a bentone, and
at least one filler, in particular a silica, advantageously treated to be lipophilic.

Preferably, the composition comprises at least colored (non-white) pigments, in particular iron oxides, and optionally additionally white pigments, as lipophilic treated pigments.

The invention will now be illustrated in the following non-limiting examples. The % are expressed as % by weight of ingredient relative to the total weight of the composition, unless otherwise indicated.

### EXAMPLES

### Example: Study of the Homogeneity of the Composition

TABLE 1

| | Control | Invention (with | |
|---|---|---|---|
| | (without | Dowsil ES-5300 | Comparative |
| | silicone | silicone | (with cetyl |
| | surfactant) | surfactant) | dimethicone) |
| Ingredients (INCI name) | % | % | % |
| C9-12 ALKANE | Qsp100 | Qsp100 | Qsp100 |
| BIS-HYDROXYPROPYL | 25 (including | 25 (including | 25 (including |
| DIMETHICONE/SMDI COPOLYMER in | 10 polymer) | 10 polymer) | 10 polymer) |
| ISODODECANE (SILMER UR-5050) | | | |
| SUNFLOWER WAX | 10 | 10 | 10 |
| CARNAUBA WAX | 1 | 1 | 1 |
| BEESWAX | 1 | 1 | 1 |
| JOJOBA ESTERS | 2 | 2 | 2 |
| SILICA | 1 | 1 | 1 |
| LAUROYL LYSINE TREATED SILICA | 3 | 3 | 3 |
| LAURYL PEG-10 | / | 1 | / |
| TRIS(TRIMETHYLSILOXY)SILYLETHYL | | | |
| DIMETHICONE AND TOCOPHEROL | | | |
| (DOWSIL ES-5300) | | | |
| CETYL DIMETHICONE (ABIL WAX | / | / | 1 |
| 9840) | | | |
| DISTEARDIMONIUM HECTORITE | 0.1 | 0.1 | 0.1 |
| TITANIUM DIOXIDE (7%) AND | 12 | 12 | 12 |
| TREATED IRON OXIDES SODIUM | | | |
| MYRISTOYL GLUTAMATE AND | | | |
| ALUMINUM HYDROXIDE (5%) | | | |
| ORGANIC PIGMENTS | 0.5 | 0.5 | 0.5 |
| CONSERVATIVES | qs | qs | Qs |
| ANTIOXIDANTS | qs | qs | Qs |

The composition is prepared according to the following procedure:
the waxes and jojoba esters are weighed and heated under rayneri at 91° C.;
when everything is homogeneous, the alkanes, the silicone surfactant and the pigments are added and left stirring for 15 minutes;
the fillers are then dispersed under rayneri and stirred for 10 minutes;
the composition is passed through the Ultra-turrax until the pigments and fillers are perfectly homogenized;
the composition is placed again under rayneri at 91° C. in order to add the Silmer UR-5050 and leave to stir for 15 minutes;
the composition is then completed with volatile linear alkanes;
the composition is hot-poured into the packaging.

The homogeneity of the compositions is observed with the naked eye, on the bulk (before pouring) and on the stick once poured.

The results are presented in the following table:

TABLE 2

| Control | Invention | Comparative |
|---|---|---|
| Presence of flow and | Homogeneous | Very slight improvement |
| marbling in the bulk | composition | but flow and marbling |
| and in the stick | Absence of flow | still present |
| | and marbling | |

These results illustrate that the presence of a (C8-C20)alkyl PEG-n Tris(trimethylsiloxy)silyl-(C1-C4)alkyl dimethicone silicone surfactant as defined in the invention allows to obtain a homogeneous composition, without flow or marbling, compared to the control composition (without surfactant) and to the composition with another silicone surfactant (cetyl dimethicone).

1. A cosmetic composition for caring for and/or making up keratin materials, comprising, in a physiologically acceptable medium:
a) a silicone-polyurethane polymer,
b) one or more pigments, advantageously treated to be lipophilic, and
c) a (C8-C20)alkyl PEG-n Tris(trimethylsiloxy)silyl-(C1-C4)alkyl dimethicone silicone surfactant
with
n ranging from 3 to 20, preferably 8 to 12,
the (C8-C20)alkyl chain being a saturated, linear or branched, hydrocarbon chain having from 8 to 20 carbon atoms, and
the (C1-C4)alkyl chain being a saturated, linear or branched, hydrocarbon chain having from 1 to 4 carbon atoms.

2. The composition according to claim 1, wherein it comprises at least non-white colored pigments, in particular iron oxides.

3. The composition according to claim 1, wherein it is an anhydrous composition.

4. The composition according to claim 1, wherein it further comprises one or more linear or branched C8-C19 alkanes.

5. The composition according to claim 1, wherein it further comprises one or more waxes.

6. The composition according to claim 5, wherein the total content of wax(es) ranges from 5 to 25% by weight relative to the total weight of the composition.

7. The composition according to claim 1, wherein the silicone-polyurethane polymer is the reaction product
of a polyorganopolysiloxane functionalized by hydroxyl groups, in particular corresponding to the structure of formula (I) 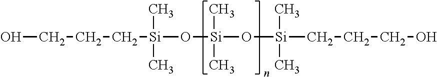
wherein n is an integer between 0 and 5000,
with a diisocyanate compound.

8. The composition according to claim 1, wherein the polyurethane silicone polymer is present in the composition in a content ranging from 3 to 25%, by weight of active material (dry matter) relative to the total weight of the composition.

9. The composition according to claim 1, wherein is the silicone surfactant Lauryl PEG-10 Tris(trimethylsiloxy)silylethyl Dimethicone.

10. The composition according to claim 1, wherein it further comprises another additional ingredient selected from lipophilic gelling agents, fillers, and mixtures thereof.

11. The composition according to claim 1, in the form of a stick or a composition packaged in a pot or a cup.

12. A cosmetic method for caring for and/or making up keratin materials, comprising the application to said keratin materials of a composition as defined in claim 1.

13. The composition according to claim 4, wherein the linear or branched C8-C19 alkanes are selected from the group consisting of branched alkanes such as isododecane, isodecane, isohexadecane, hemisqualane; linear C8-C19 alkanes such as nonane, dodecane, undecane, tridecane, n-tetradecane, n-pentadecane, n-nonadecane and mixtures thereof.

14. The composition according to claim 5, wherein it further comprises one or more pasty fatty substances.

15. The composition according to claim 7, wherein the silicone-polyurethane polymer is the reaction product
of a polyorganopolysiloxane functionalized by hydroxyl groups, corresponding to the structure of formula (I) 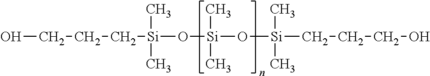
wherein n is an integer between 10 and 100,
with a diisocyanate compound selected from the group consisting of 1,6-hexamethylene diisocyanate, dicyclohexylmethane 4,4′-diisocyanate, isophorone diisocyanate, and combinations thereof.

16. The composition according to claim 11, which is a lip stick, a liner pencil, an eyebrow pencil, an eye shadow, or a concealer.

17. The cosmetic method according to claim 12 for caring for and/or making up the skin, eyelids, eyebrows or lips.
